(19) United States
(12) Patent Application Publication
(10) Pub. No.: US 2016/0355847 A1
(43) Pub. Date: Dec. 8, 2016

(54) Novel Clostridium Species That Converts Wheat Straw and Switchgrass Hydrolysates Into Butyric Acid

(71) Applicant: The United States of America, as Represented by the Secretary of Agriculture, Washington, DC (US)

(72) Inventors: Siqing Liu, Dunlap, IL (US); Kenneth M. Bischoff, Morton, IL (US); Timothy D. Leathers, Dunlap, IL (US); Joseph O. Rich, Morton, IL (US)

(21) Appl. No.: 15/173,602

(22) Filed: Jun. 3, 2016

Related U.S. Application Data
(60) Provisional application No. 62/170,499, filed on Jun. 3, 2015.

(51) Int. Cl. C12P 7/52; C12N 1/22; C12R 1/145

(52) U.S. Cl. CPC C12P 7/52; C12R 1/145; C12N 1/22

(57) ABSTRACT

The present disclosure is directed to methods for producing butyric acid comprising fermenting a lignocellulosic biomass hydrolysate using C. tyrobutyricum strain RPT-4213 under anaerobic conditions using dilute acid-pretreated hydrolysates of wheat straw, corn fiber, corn stover, rice hull, and switchgrass, for example.

### REFERENCE TO RELATED APPLICATION

This application claims the benefit of U.S. Provisional Application No. 62/170,499, filed Jun. 3, 2015, which is incorporated herein by reference in its entirety.

### FIELD OF THE INVENTION

The invention relates to a composition comprising Clostridium tyrobutyricum RPT-4213, method for producing butyric acid from glucose using a novel microorganism C. tyrobutyricum strain RPT-4213, and a system for producing butyric acid from lignocellulosic biomass hydrolysates using strain RPT-4213.

### BACKGROUND OF THE INVENTION

Fermentation processes using microorganisms provide a promising path for converting biomass and agricultural wastes into chemicals and fuels. Hemicellulose biomass contains an abundance of pentose sugars that can be used to produce chemicals and fuels.

Butyric acid (C₃H₇COOH) is a short-chain fatty acid with a pKa of 4.82. Pure butyric acid is a clear, foul-smelling liquid, while esters of butyric acid can be found naturally in animal fat and some plant oils. Esters and salts of butyric acid have been used in animal feeds and as a flavoring in various food products. Other applications of butyric acid include varnishes, perfumes, pharmaceuticals and disinfectants, as well as for the production of plastics, plasticizers, surfactants, and textile auxiliaries (Zigova et al., Process Biochemistry, Volume 34, 835-843, 1999). Butyric acid is also useful for the production of butanol. Butyric acid and hydrogen are converted to butanol through catalytic hydrogenation or hydrogenolysis of butyrate ester.

Butyric acid can be synthesized by either a petrochemical or a biological route (Jones and Woods, Microbiological Reviews, Volume 50, 484-524, 1986). Currently, commercial production from petroleum feedstocks has dominated the butyric acid market. However, due to limited resources, there has been a growing interest in the production of butyric acid via microbial fermentation from renewable feedstocks. In addition, the biological production of butyric acid addresses sustainability concerns and may satisfy consumer preferences in applications such as food additives or cosmetic products.

Gram-positive, obligate anaerobic bacteria species, including Butyrivibrio fibrisolvens, Clostridium acetobutylicum, Clostridium aurantibutyricum, Clostridium beijerinckii, Clostridium butyricum, Clostridium kluyveri, Clostridium pasteurianum, Clostridium tenanomorphum, Clostridium tyrobutyricum, have been described as producing butyric acid in significant quantities (Jones and Woods, 1986, supra McCoy et al., Journal of Infectious Diseases, Volume 46, 118-137, 1930; and Murty and Chandra, Antonie van Leeuwenhoek, Volume 55, 153-163, 1989). Among these microorganisms, most research has employed either C. acetobutylicum or C. tyrobutyicum for butyric acid fermentation (Barbeau et al., Applied Microbiology and Biotechnology, Volume 29,447-455, 1988; Fayolle et al., Journal of Industrial Microbiology, Volume 6, 179-183, 1990; Jiang et al., Applied Biochemistry and Biotechnology, Volume 160, 350-359, 2010; Michel-Savin et al., Applied Microbiology and Biotechnology, Volume 33, 127-131, 1990a, Michel-Savin et al., Applied Microbiology and Biotechnology, Volume 32, 387-392, 1990b; Wu and Yang, Biotechnology and Bioengineering, Volume 82, 93-102, 2003; Zhu et al, Process Biochemistry, Volume 38, 657-666, 2002; Zhu and Yang, Biotechnology Progress, Volume 19, 365-372, 2003, Zhu and Yang, Journal of Biotechnology, Volume 110, 143-157, 2004). The metabolic pathways for production of butyric acid from glucose have been described (Girbal et al., FEMS Microbiology Reviews, Volume 17, 287-297, 1995; Janati-Idrissi et al., Biochemistry and Cell Biology, Volume 67, 735-739, 1989; Monot et al., Applied Microbiology and Biotechnology, Volume 19, 442-426, 1984), which involve the breakdown of hexose to pyruvate and then acetyl-CoA via the Embden-Myerhof-Parnas pathway. Two analogous branches from acetyl-CoA produce either acetate or butyrate. Acetic acid is produced from acetyl-coA via phosphotransacetylase and acetate kinase, while butyric acid is produced from acetyl-CoA derived acetoacetyl CoA via phosphotransbutyrylase and butyrate kinase (Liu et al., Biotechnology Progress, Volume 22, 1265-1275, 2006).

Both acetic acid and butyric acid can be inhibitory to C. acetobutylicum in batch fermentations (Michel-Savin et al., 1990a, supra). To overcome this barrier, new fermentation processes such as the fibrous bed bioreactor (FBB) have been studied. The immobilized-cell fermentation in the FBB system showed improved butyric acid tolerance and productivity (Jiang et al., 2010 supra; Wu and Yang, 2003 supra; Zhu and Yang, 2003, supra). More recently, with repeated fed-batch fermentation in nutrient media and glucose, an adapted C. acetobutylicum strain was reported to accumulate up to 86.9 g L⁻¹ (Jiang et al., Biotechnology and Bioengineering, Volume 108, 31-40, 2011).

Other than improving fermentation technologies such as applications of the FBB system, new and improved strains were sought for butyric acid production. For example, C. butyricum strain ZJUCB strain was reported for butyric acid production (He et al., Journals of Zhejiang University Science, Volume B6, 1076-1080, 2005). In a pH-controlled batch fermentation, a butyric acid titer reached 12.25 g L⁻¹ at pH 6.5. When this strain was used in a fed-batch fermentation, butyric acid production increased to 16.74 g L⁻¹ (He et al., supra).

Still, the butyric acid fermentation process is not economically competitive or sustainable because of the use of nutrient media. A limited number of studies have been reported for sustainable production of butyric acid from pretreated lignocellulosic biomass hydrolysate. One study used the fibrous bed bioreactor system to convert corn fiber hydrolysate to butyric acid with a productivity of 2.9 g/L/h (Zhu et al., 2002, supra). More recently, Wei et al., (Bioresource Technology, Volume 129, 553-560, 2013) reported butyric acid productivity of 0.51 g/L/h and a yield of 0.45 g/g sugar using the fibrous bed bioreactor system and media containing 15-20% sugarcane bagasse hydrolysate. When dilute sulfuric acid-pretreated cane molasses was used as substrate, batch fermentations produced 26.2 g/L butyrate at pH 6.0, while repeated-batch and fed-batch fermentations resulted in 34.6 g/L and 55.2 g/L, respectively (Jiang et al., Bioresource Technology, Volume 100, 3403-3409, 2009). Jerusalem artichoke was tested as another potential low valued feedstock for butyric acid production by Huang et al (Bioresource Technology, Volume 102, 3923-3926, 2011). In this study the butyric acid yield was 0.44g/g with a productivity of 2.75 g/L/h using the fibrous bed bioreactor and C. tyrobutyricum (ZJU 8235). More research is needed for profitable production at the industrial scale.

Thus, what is needed in the art is a composition, system and method for producing butyric acid from renewable resources such as lignocellulosic biomass hydrolysates in a fermentation system that utilizes glucose in a cost-effective culture medium. Fortunately, as will be clear from the following disclosure, the present invention provides for this and other needs.

### SUMMARY OF THE INVENTION

It is therefore an object of the present invention to provide a novel composition having C. tyrobutyricum strain RPT-4213 in a fermentation medium that produces butyric acid when fermented on a lignocellulosic biomass hydrolysate.

Another object of the present invention is to provide a fermentation system for producing butyric acid that includes C. tyrobutyricum and a lignocellulosic biomass.

Another object of the present invention is to provide a novel method of producing butyric acid using C. tyrobutyricum strain RPT-4213 and lignocellulosic biomass hydrolysate in a fermentation process.

A still further object of the present invention is to provide a method for producing butyric acid in a fermentation process using C. tyrobutyricum strain RPT-4213 and a lignocellulosic biomass hydrolysate selected from the group consisting of wheat straw hydrolysate, corn fiber hydrolysate, corn stover hydrolysate, rice hull hydrolysate, and switchgrass hydrolysate.

Other features, objects and advantages of the invention will be apparent from the detailed description which follows.

### Deposit of the Microorganisms

C. tyrobutyricum RPT-4213, designated NRRL B-67062, was deposited on May 28, 2015. It was deposited under the provisions of the Budapest Treaty, on the International Recognition of the Deposit of Microorganisms for the Purposes of Patent Procedure and the Regulations there under (Budapest Treaty) with the U.S.D.A. Agricultural Research Service Patent Culture Collection, National Center for Agricultural Utilization Research, 1815 N. University Street, Peoria, Ill. 61604. Access to this deposit will be available during the pendency of the application to the Commissioner of Patents and Trademarks and persons determined by the Commissioner to be entitled thereto upon request. Upon allowance of any claims in the application, the Applicant(s) will maintain and will make this deposit available to the public pursuant to the Budapest Treaty.

### BRIEF DESCRIPTION OF THE DRAWINGS

FIG. 1 shows a phylogenetic relationships of RPT-4213 to several closely related Clostridia based on 16S rDNA sequences. Genbank accession numbers are provided in parentheses. C. tyrobutyricum (L08062.1), C. ragsdalei (65306484), C. ljungdahlii (300433347), C. kluyveri (152206095), C. tunisiense (28207688), C. thiosulforeducen (13506625), C. subterminale (281485351), C. acidisoli (7327846), C. pasteurianum (148887529), C. acetobutylicum (NC_003030), C. perfringens (NC_003366), C. aciditolerans (72199308), C. scatologenes (333777478), and C. drakei (21684961).

FIG. 2A-2F are graphs showing amounts of glucose, acetic acid, and butyric acid in fermentations using wheat straw hydrolysate (WSH). Approximately 5 mL culture fermentations using C. tyrobutyricum strain RPT-4213 and varying concentrations of WSH and Ct basal medium were carried out for approximately 144 hours. The production of close to baseline level of acetic acid was not presented in here. FIG. 2A shows amounts of glucose, acetic acid, and butyric acid produced in fermentation using a concentration of approximately 30% WSH and approximately 1× Ct; FIG. 2B shows amounts of glucose, acetic acid, and butyric acid produced in fermentation using a concentration of approximately 30% WSH and approximately 0.5× Ct, FIG. 2C shows amounts of glucose, acetic acid, and butyric acid produced in fermentation using approximately 40% WSH and approximately 1× Ct, FIG. 2D shows amounts of glucose, acetic acid, and butyric acid produced in fermentation using approximately 40% WSH and approximately 0.5× Ct; FIG. 2E shows amounts of glucose, acetic acid, and butyric acid produced in fermentation using approximately 50% WSH and approximately 1× Ct; and FIG. 2F amounts of glucose, acetic acid, and butyric acid produced in fermentation using approximately 50% WSH and approximately 0.5× Ct.

FIG. 3A-3F are graphs showing amounts of glucose, acetic acid, and butyric acid in fermentations using switch grass hydrolysate (SGH). Approximately 5 mL culture fermentation using C. tyrobutyricum strain RPT-4213 and varying concentrations of SGH and Ct basal medium were carried out for approximately 144 hours. Approximately 50% SGH hydrolysate supplemented with approximately 1× Ct (bottom left panel) produced approximately 6.01 g/L butyric acid and approximately 50% SGH and 0.5× Ct (bottom right) produced approximately 5.91 g/L butyric acid. FIG. 3A shows amounts of glucose, acetic acid, and butyric acid produced in fermentation using a concentration of approximately 30% SGH and approximately 1× Ct; FIG. 3B shows amounts of glucose, acetic acid, and butyric acid produced in fermentation using a concentration of approximately 30% SGH and approximately 0.5× Ct, FIG. 3C shows amounts of glucose, acetic acid, and butyric acid produced in fermentation using approximately 40% SGH and approximately 1× Ct, FIG. 3D shows amounts of glucose, acetic acid, and butyric acid produced in fermentation using approximately 40% SGH and approximately 0.5× Ct; FIG. 3E shows amounts of glucose, acetic acid, and butyric acid produced in fermentation using approximately 50% SGH and approximately 1× Ct; and FIG. 3F amounts of glucose, acetic acid, and butyric acid produced in fermentation using approximately 50% SGH and approximately 0.5× Ct.

FIG. 4A-4F are graphs showing amounts of glucose, acetic acid, and butyric acid in fermentations using corn stover hydrolysate (CSH). Approximately 5 mL culture fermentation using C. tyrobutyricum strain RPT-4213 and varying concentrations of CSH and Ct basal medium were carried out for approximately 144 hours. Approximately 50% CSH hydrolysate supplemented with approximately 1× Ct (bottom left panel) or approximately 0.5× Ct (bottom right panel) produced approximately 1.32 g/L or 0.85 g/L butyric acid. Approximately 40% CSH and 1× Ct (middle left panel) produced approximately 4.24 g/L butyric acid. FIG. 4A shows amounts of glucose, acetic acid, and butyric acid produced in fermentation using a concentration of approximately 30% CSH and approximately 1× Ct; FIG. 4B shows amounts of glucose, acetic acid, and butyric acid produced in fermentation using a concentration of approximately 30% CSH and approximately 0.5× Ct, FIG. 4C shows amounts of glucose, acetic acid, and butyric acid produced in fermentation using approximately 40% CSH and approximately 1× Ct, FIG. 4D shows amounts of glucose, acetic acid, and butyric acid produced in fermentation using approximately 40% CSH and approximately 0.5× Ct; FIG. 4E shows amounts of glucose, acetic acid, and butyric acid produced in fermentation using approximately 50% CSH and approximately 1× Ct; and FIG. 4F amounts of glucose, acetic acid, and butyric acid produced in fermentation using approximately 50% CSH and approximately 0.5× Ct.

FIG. 5A-5F are graphs showing amounts of glucose, acetic acid, and butyric acid in fermentations using corn fiber hydrolysate (CFH). Approximately 5 mL culture fermentation using C. tyrobutyricum strain RPT-4213 and varying concentrations of CFH and Ct basal medium were carried out for approximately 144 hours. Approximately 50% CFH produced approximately 6.45 g/L butyric acid. FIG. 5A shows amounts of glucose, acetic acid, and butyric acid produced in fermentation using a concentration of approximately 30% CFH and approximately 0.5× Ct, FIG. 5C shows amounts of glucose, acetic acid, and butyric acid produced in fermentation using approximately 40% CFH and approximately 1× Ct. FIG. 5D shows amounts of glucose, acetic acid, and butyric acid produced in fermentation using approximately 40% CFH and approximately 0.5× Ct; FIG. 5E shows amounts of glucose, acetic acid, and butyric acid produced in fermentation using approximately 50% CFH and approximately 1× Ct; and FIG. 5F amounts of glucose, acetic acid, and butyric acid produced in fermentation using approximately 50% CFH and approximately 0.5× Ct.

FIGS. 6A and 6B are graphs showing bioreactor fermentation showing amounts of glucose, acetic acid and butyric acid using approximately 60% biomass hydrolysate from wheat straw (FIG. 6A-top) and switchgrass (FIG. 6B-bottom) with approximately 0.5× Ct and controlled pH of approximately 6.0.

### DETAILED DESCRIPTION OF THE INVENTION

### Definitions

As used herein, the term “acid” such as butyric acid, is intended to encompass free acid and salts which are generated by combination of such acids and suitable bases which may be present in the medium during the process, for example, butyric acid may include free butyric acid, sodium butyrate, ammonium butyrate, lithium butyrate, calcium butyrate, potassium butyrate, magnesium butyrate, ammonium butyrate, and aluminum butyrate, for example. In addition, the terms butyric acid and butyrate are interchangeable to describe the acid or its deprotonated form, depending on the pH value of its environment. The same applies to other acids, for example lactic acid/lactate, propionic acid/propionate, and acetic acid/acetate. Unless otherwise specified, chemical names described herein refer to all isomeric forms of the chemical names, such as enantiomers, diastereomers, and conformation isomers. For example, the term lactic acid, glucose, xylose, galactose refers to both D and L isomers. When the carbohydrate is capable of existing in both open-chain and cyclic form, both alpha and beta isomers of the chair form are encompassed. As used herein, the terms fermenting and fermentation refer to a process in which microorganisms such as bacteria, yeast, and other small organisms metabolize one or more substances to produce energy and chemicals needed to live and reproduce. This process of chemical reactions will produce some forms of by-product. Microorganisms are capable of generating a wide array of molecules as end points to fermentation. Fermentation is an ATP-generating process in which organic compounds act as both donors and acceptors of electrons and it can take place in the absence of oxygen (Berg et al., Biochemistry, Chapter 16, 2002).

As used herein, the term feedstock, biomass hydrolysate, fermentation medium is the raw material for fermentation. The fermentation medium is any medium suitable for fermentation and/or for growth of C. tyrobutyricum. It may further comprise fermentable substrates. The biomass hydrolysate is a lignocellulosic biomass and is generally composed of lignocelluloses which is a complex consisting of cellulose, hemicelluloses, lignin, etc. although the chemical composition and content may vary depending on the plant from which it is derived. The substrates include lignocellulosic hydrolysates including but not limited to, corn stover hydrolysate, corn fiber hydrolysate, switchgrass hydrolysate, rice hull hydrolysate, and wheat straw hydrolysate. Other substances may also be present in the medium as needed. For example, NaOH, NH₄OH, NaH₂PO₃, Na₂HPO₃, citric acid, HCl, NH₄Cl may be added to adjust the pH value of the feedstock to a desired value, for example, pH 6, or to adjust other physical, chemical, or physiological properties.

As used herein, the term microorganism refers to a living organism too small to be seen with the naked eye including bacteria, fungi, protozoans, algae, and viruses.

As used herein, a strain of a bacterium may be a wild-type strain or a mutant strain. Inoculum size (w/v) refers to the ratio of the weight of the cells to the relative total volume of feedstock.

As used in the specification and claims the singular form “a”, “an”, and “the” include plural references unless the context clearly dictates otherwise. For example, the term “a bacterium” includes a plurality of microorganisms of the same strain.

As used in the specification and claims, the terms “about” and “approximately” mean to be nearly the same as a referenced number or value. As used herein, the terms “about” and “approximately” should be generally understood to encompass ±10% of a specified amount, frequency or value. Further, all numbers expressing the quantities used the specification and claims, for example, concentrations, reaction conditions, time, temperature and yield, are modified by the term “approximately” unless otherwise indicated. As used herein, when a numerical range is given, both ends of the range are included.

The term substantial or substantially mean of real worth or importance, or considerable value For example, a substantial increase or decrease means a change greater than 5% of the previous measured value.

### Introduction:

A novel anaerobic C. tyrobutyricum strain RPT-4213 has been discovered and identified. This strain ferments glucose in various media such as MRS (Becton Dickinson, Sparks, Md.), Ct (Fayolle et al., 1990, supra). Cz (Wu et al., Journal of Biotechnology, Volume 165, 18-21, 2013), and TGY broth (Wu et al., Journal of Biotechnology, Volume 165, 18-21, 2013) and produces butyric acid in a high yield of approximately 0.48 g/gram of glucose which is a yield of approximately 97.9% of the theoretical yield of approximately 0.49 g butyric acid/g glucose in MRS medium. Ct medium produces a slightly less amount of butyric acid and is the preferred medium because it is less costly to use. The strain can tolerate inhibitors released from dilute acid pretreated lignocellulosic biomass hydrolysates. This strain of C. tyrobutyricum fermented glucose to butyric acid and a small amount of acetic acid but does not utilize five carbon sugars such as xylose and arabinose.

The present invention includes a RPT-4213 seed culture-containing composition. The composition includes C. tyrobutyricum RPT-4213 in a culture medium. A typical fermentation contains 300 ml fermentation broth including 90 -180 ml biomass hydrolysate (30-60%), 15-30 ml Ct media (0.5×-1×), 30 ml of a seed culture of C. tyrobutyricum RPT-4213 having an OD600 of approximately 4-7 and X ml sterile water to make up the final volume.

A biomass feedstock with fermentable carbohydrates is fed to a fermentation bioreactor, such as those manufactured by Applikon, B. Braun, DasGip, and New Brunswick Scientific, for butyric acid fermentation by butyric acid producing C. tyrobutyricum strain RPT-4213 at mildly elevated temperatures, typically about 37 degrees C. The hydrolysate is prepared by treating a ground up biomass with acid, heat and enzymes as described below in the Example 2. The fermentation is allowed to incubate for approximately 144 hrs with controlled pH (6.0) using 4 M NaOH and 4 M phosphoric acid, and approximately 100 rpm agitation with an initial nitrogen purge. Anaerobic conditions were maintained by sparging with nitrogen every 24 hrs. For commercial scaled fermentation, a continuous bioreactor fermentation is used and the butyric acid is purified from the fermentation broth by solvent extraction and NaOH striping (Wu and Yang Biotechnology and Bioengineering Volume 82, 93-102, 2003)

The following examples are intended only to further illustrate the invention and are not intended to limit the scope of the invention as defined by the claims.

### EXAMPLES

### Example 1

### Bacterial Strains and Growth Conditions

C. tyrobutyricum strain RPT-4213 was grown on MRS agar plates (Becton Dickinson, Sparks, Md,) under anaerobic conditions at approximately 37 degrees C. A single colony of RPT-4213 was inoculated in approximately 3 mL MRS broth and grown for approximately 24-48 hours in an anaerobic jar at about 37 degrees C. Active cultures of RPT-4213 were maintained by transferring about 0.1% inocula to fresh MRS medium on a weekly basis.

This strain was tested for growth and fermentation in MRS and three media previously described for Clostridia. Fayolle et al. (1990, supra) described a medium which is described in this specification as Ct which contains the following: Glucose, approximately 20 g/L; yeast extract, approximately 5.0 g/L; (NH₄)₂SO₄, approximately 1.0 g/L; K₂HPO₄, approximately 1.5 g/L; MgSO₄.7H₂O, approximately 0.6g/L; FeSO₄.7H₂O, approximately 0.03 g/L.

Cultures of RPT-4213 were cleared of bacterial cells by centrifugation and the supernatant liquid filtered through a 0.2 μm filter (Nalgene). About 300 μl of the filtered supernatant was analyzed by HPLC to identify and quantify fermentation products (Liu et al., Journal of Industrial Microbiology and Biotechnology, Volume 35, 75-81, 2008).

Genomic DNA was isolated using a Gram-positive genomic DNA isolation kit following the manufacturer's instructions (Epicentre, Madison, Wis.) (Liu et al., Journal of Bacteriology, Volume 193, 4019-4020, 2011). Genomic PCRs were performed using 16S rDNA primers (Whitehead and Cotta, Current Microbiology, Volume 43, 17-20, 2001), and the product was sequenced using the ABI prism DNA dye terminator cycle sequencing ready reaction kit and the ABI prism 310 DNA sequencer (Perkin-Elmer, Foster City, Calif.). Sequence analyses were carried out with the San Diego Supercomputer Center Biology Workbench (http://www.sdsc.edu/Research/biology/) and through the National Center for Biotechnology Information, NCBI (http://www.ncbi.nim.nih.gov/).

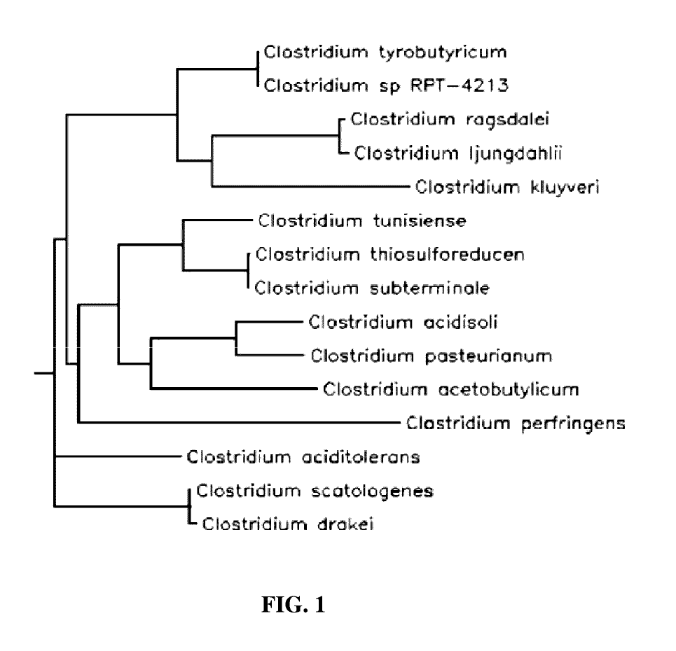

Fifty bacterial isolates from fuel ethanol production plants (Liu et al, 2008, supra) were grown individually from single colonies in approximately 3 mL cultures in MRS broth for approximately 24-48 hours in an anaerobic jar at about 37 degrees C., and used for fermentation screening analyses. Strain RPT-4213 was distinguished by the production of butyric acid. RPT-4213 was identified as a strain of C. tyrobutyricum by 16S rDNA sequencing (GenBank accession number KC692790.1). FIG. 1 shows a phylogenic tree generated by CLUSTALW program using the SDSC biology workbench (http://workbench.sdsc.edu/). The deposited RPT-4213 sequence shared 100% identities with that of C. tyrobutyricum NIZO 51 (GenBank accession number L08062.1) and showed a score of 1629 with 300433347 (Clostridium ljungdahlii DSM 13528) and 65306484 (Clostridium ragsdalei).

RPT-4213 only grows under strictly anaerobic conditions, and once the culture was established, it was maintained by weekly transfer into fresh MRS medium under anaerobic conditions.

C. tyrobutyricum strain RPT-4213 was first tested for utilization of glucose and other substrates in approximately 5 ml cultures containing MRS broth and three other media previously described for growth of Clostridium (Fayolle et al., 1990, supra and Wu et al., 2013, supra). The strain fermented glucose to butyric acid and a small amount of acetic acid in all media tested (Table 1). However, it did not utilize five carbon sugars such as xylose and arabinose (data not shown). In MRS medium containing approximately 19.74 g/L glucose, the strain produced approximately 9.47 g/L butyric acid in about 48 hours, resulting in a yield of approximately 0.48 g/g glucose, which is approximately 97.9% of the theoretical yield of approximately 0.49 g/g of butyric acid /glucose). In MRS medium containing a mixture of glucose and xylose, only the glucose was utilized (Table 1). Fermentations using the less complicated and less expensive Ct medium resulted in a yield of approximately 0.46 g butyric acid/g glucose (Table 1). Ct media was chosen to replace MRS media as the supplemental nitrogen and iron source for fermentation of biomass hydrolysates.

TABLE 1
Evaluation of fermentation media for production of butyric
acid by C. tyrobutyricum strain RPT-4213.

| | | | | | | | Butyric |
|---|---|---|---|---|---|---|---|
| | | | | | | Bulyric | Acid/ |
| Growth | Time | Glucose | Xylose | Lactate | Acetate | Acid | glucose |
| Media | (h) | g/L | g/L | g/L | g/L | g/L | g/g |
| MRS | 0 | 19.74 | | 0.04 | 4.39 | 0.00 | |
| | 48 | 0.04 | | 0.00 | 4.58 | 9.47 | 0.48 |
| Ct | 0 | 20.31 | | 0.00 | 0.00 | 0.00 | |
| | 48 | 0.00 | | 0.40 | 0.20 | 9.26 | 0.46 |
| Cz | 0 | 23.10 | | 0.00 | 3.54 | 0 | |
| | 48 | 0.00 | | 0.00 | 3.58 | 10.40 | 0.45 |
| MRS | 0 | 20.11 | 10.73 | 0.03 | 4.55 | 0.16 | |
| 1% | | | | | | | |
| Xylose | | | | | | | |
| | 48 | 0.00 | 10.54 | 0.00 | 4.73 | 8.76 | 0.44 |
| TGY | 0 | 19.27 | | 0.12 | 0.12 | 0.46 | |
| | 48 | 0.02 | | 000 | 0.76 | 8.00 | 0.39 |

### Example 2

### Biomass Hydrolysates

Agricultural biomass residues including corn stover, corn fiber, wheat straw, rice hull, and the energy crop switchgrass were obtained from USDA, Peoria. The moisture content of each biomass material was determined and used to calculate the dry mass. These lignocellulosic biomass materials were milled into fine particles using a hammer mill prior to pretreatment as described previously (Qureshi et al., Bioprocess and Biosystems Engineering, Volume 30, 419-427, 2007, herein incorporated by reference in its entirety). Substrate powders were made into 10% (w/v) slurries with approximately 0.5% H₂SO₄ and heated under conditions previously determined for each substrate. Corn fiber, rice hull, and switchgrass (Qureshi et al., Biomass and Bioenergy, Volume 34, 566-571, 2010; Saha et al., Biotechnology Progress, Volume 21, 816-822, 2005) were pretreated at approximately 160 degrees C. for about 10 minutes in an IR Dyer Machine. Corn stover slurries were similarly heated for about 20 minutes. Wheat straw slurries (Qureshi et al., 2007, supra) were pretreated by autoclaving at approximately 121 degrees C. for about 60 minutes. After cooling, approximately 10M NaOH was used to adjust the slurries to pH 5.0, and the slurries were diluted to approximately 5% (w/v).

A mixture of enzymes was added to complete the hydrolysis as described (Qureshi et al., 2007, supra). For corn fiber, rice hull, and switchgrass, approximately 6 mL of each of cellulase (Celluclast 1.51, Sigma-Aldrich, St. Louis, Mo.), beta-glucosidase (Novozyme 188, Sigma-Aldrich), and xylanase (FiberZyme LBR, Dyadic International, Jupiter, Fla.), were added to approximately 1 liter of the dilute acid pretreated slurries and the material was incubated at approximately 45 degrees C. for about 75 hours at about 150 rpm. Wheat straw and corn stover were similarly treated using a mixture of Celluclast 1.5L, Novozyme 188, and Viscostar 150 L xylanase (Dyadic). Hydrolysates were then clarified by centrifugation and the supernatants were sterilized using a 0.2 μm filter and adjusted to pH approximately 6.0 with approximately 3 M KOH. The sugars and inhibitors released were determined by HPLC. The sterilized solutions were aliquoted and stored at about −20 degrees C.

Corn fiber, rice hull, switchgrass, wheat straw, and corn stover were hydrolysed using a dilute acid pretreatment followed by enzyme treatment. Pretreatment parameters, including temperatures, time, and the type and dose of hydrolytic enzymes were tailored for each biomass feedstock based on previous studies (Avci et al., Bioresource Technology, Volume 130, 603-612,2012; Saha et al., 2005, supra; Qureshi et al., 2007, 2010, supra; all incorporated by reference in their entirety). Hydrolysates were adjusted to approximately 5% (w/v) and clarified before analysis for glucose and fermentation inhibitors (Table 2). Simple sugars, including glucose, xylose, and arabinose, were released by enzymatic hydrolysis but only glucose is presented here since the strain cannot use xylose and arabinose. Corn fiber and wheat straw hydrolysates contained the highest levels of glucose at approximately 35-39 mg/mL (Table 2). Rice hull hydrolysate contained the lowest concentration of glucose at approximately 11 mg/mL. The fermentation inhibitors acetic acid, HMF, and furfural also were produced during the pretreatment process. The order of HMF production relative to gram of glucose released from high to low is as follows: (CSH)>(SGH)>(RHH)>(CFH)>(WSH), and the amount of furfural released relative to per gram of glucose released ranges from high to low: RHH>CFH>SGH>CSH>WSH. The results shown in Table 2 indicate that among the five types of biomass, dilute acid pretreated wheat straw hydrolysate contains the lowest amount of inhibitors per glucose and rice hull hydrolysate contains the highest amount of furfural per glucose (μM/g). It is known that the rice plants accumulate up to 10% silica in the straw at maturation stage, which might be a factor in the reduced efficiency (lowest amount of glucose released) from the dilute acid pretreatment used in these examples.

TABLE 2
Glucose and inhibitor concentrations of five different biomass
hydrolysates

| | | Lactic | Acetic | | | HMF/ | Furfural/ |
|---|---|---|---|---|---|---|---|
| | Glucose | acid | Acid | HMF | Furfural | glucose | glucose |
| Samples | mg/ml | mg/ml | mg/ml | mM | mM | μM/g | μM/g |
| Corn | 38.63 | 0.47 | 3.08 | 1.95 | 8.04 | 50.48 | 208.13 |
| Fiber | | | | | | | |
| Rice | 10.96 | 0.18 | 3.25 | 1.32 | 5.28 | 120.44 | 481.75 |
| Hull | | | | | | | |
| Switch- | 27.50 | 0.15 | 3.34 | 6.75 | 4.94 | 245.46 | 179.64 |
| grass | | | | | | | |
| Wheat | 35.36 | 0.14 | 3.05 | 1.27 | 3.44 | 35.64 | 96.55 |
| Straw | | | | | | | |
| Corn | 26.36 | 0.17 | 3.86 | 8.75 | 4.12 | 331.94 | 156.30 |
| Stover | | | | | | | |

### Example 3

### Fermentation for Butyric Acid Product

Small scale cultures of approximately 5 mL in 14 mL culture tubes, were incubated statically without pH control at about 37 degrees C. under anaerobic conditions, inoculated to approximately 5% (v/v) with a freshly grown overnight preinoculum having an OD₆₀₀ of approximately 4-7. Initial experiments compared 2% (w/v) glucose with or without 1% (w/v) xylose in four basal media: MRS, Ct, Cz, or TGY (Table 1). Biomass substrates corn stover hydrolysate (CSH), corn fiber hydrolysate (CFH), switchgrass hydrolysate (SGH), rice hull hydolysate (RHH), or wheat straw hydrolysate (WSH) were similarly tested at approximately 30-50% (w/v) in Ct basal medium or diluted (0.5×) Ct medium (Table 3). Culture samples of approximately 300 μL were centrifuged at about 13,000 rpm for an amount of time to produce a useable supernatant and said supernatants were used for GC and HPLC analyses.

Bioreactor cultures were in 500 mL vessels (DASGIP Parallel Bioreactor Systems, Germany). A typical fermentation contains 300 ml fermentation broth including 90-180 ml biomass hydrolysate (30-60%), 15-30 ml Ct media (0.5×-1×), 30 ml of a seed culture of C. tyrobutyricum RPT-4213 having an OD600 of approximately 4-7 and sterile water to make up the final volume. Fermentations were carried out at approximately 37 degrees C. with controlled pH (6.0) and about 100 rpm agitation with an initial nitrogen purge. Anaerobic conditions were maintained by sparging the cultures with nitrogen. The samples were collected periodically up to about 144 hours to measure the OD600 in a spectrophotometer (Biomate™3, Thermo Spectronic) and the residual substrates and newly formed products were monitored by HPLC analyses as described below. All fermentation experiments were performed in duplicate.

Fermentation products including butyric acid and butanol were analyzed by gas chromatography (6890N; Agilent Technologies, Wilmington, Del., USA) equipped with a hydrogen flame ionization detector operated at about 250 degrees C. and packed isothermal capillary column operated at about 120 degrees C. as described previously (Qureshi et al., 2007, supra). Prior to injection into the GC the samples were diluted approximately 4 fold with distilled water.

Carbohydrates and fermentation products, including lactate, acetate, and butyric acid, were analyzed by HPLC (Agilent 1100 series, Agilent Technologies, New Castle, Del.) as described previously (Liu et al., 2008, supra). The system used a 300 mm Aminex HPX-87H column (BioRad, Richmond, Calif.) and a refractive index detector (G1362A, Agilent Technologies, Palo Alto, Calif.). Samples were run at about 65 degrees C. and eluted at approximately 0.6 mL/minute with approximately 5 mM sulfuric acid. Fermentation inhibitors including furfural and 5-hydroxymethylfurfural (HMF) were determined by HPLC with a UV detector at approximately 277 nm. Samples were run at ambient temperature on an Econosphere C18 column (5 μm, 250×4.6 mm, Alltech, Deerfield, Ill.) and eluted with acidified methanol as described (Nichols et al., Enzyme and Microbial Technology, Volume 42 (7), 624-630, 2008). All analytical determinations were done in duplicate and average results shown.

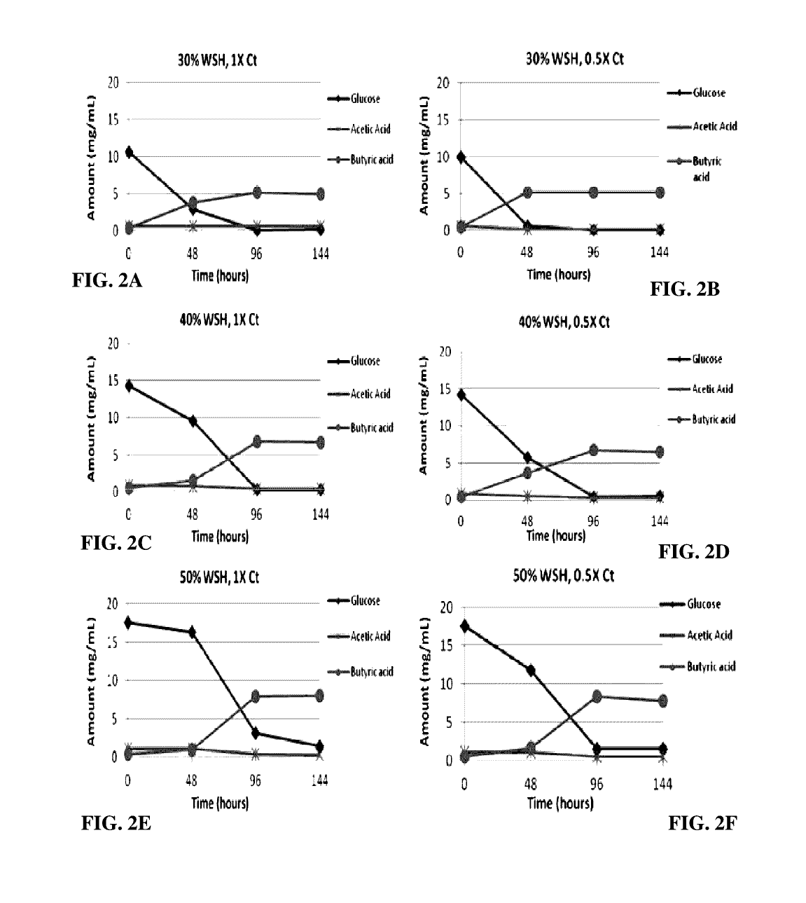

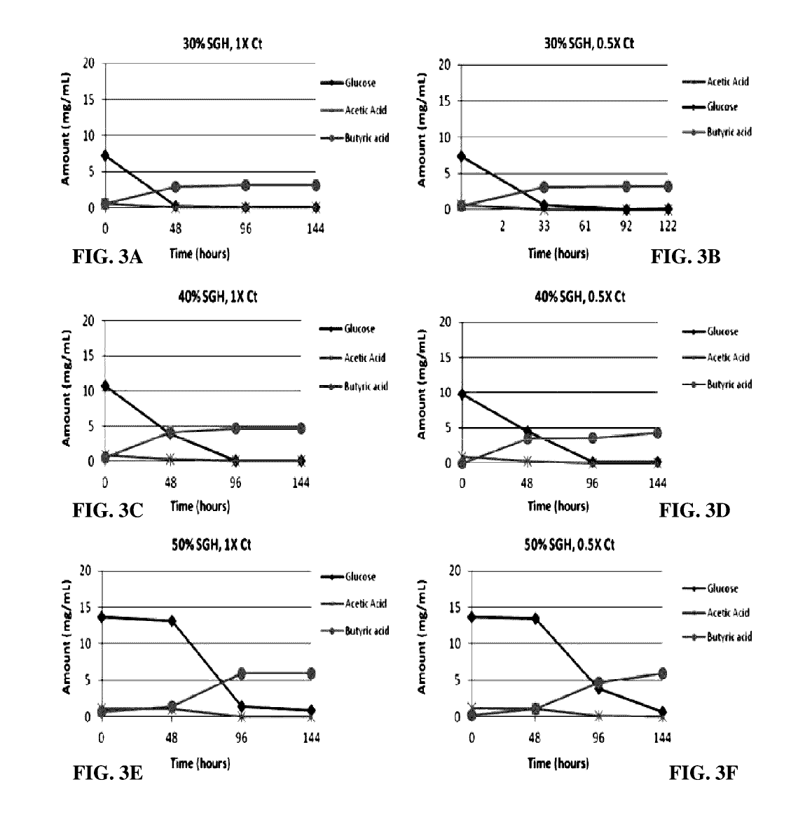

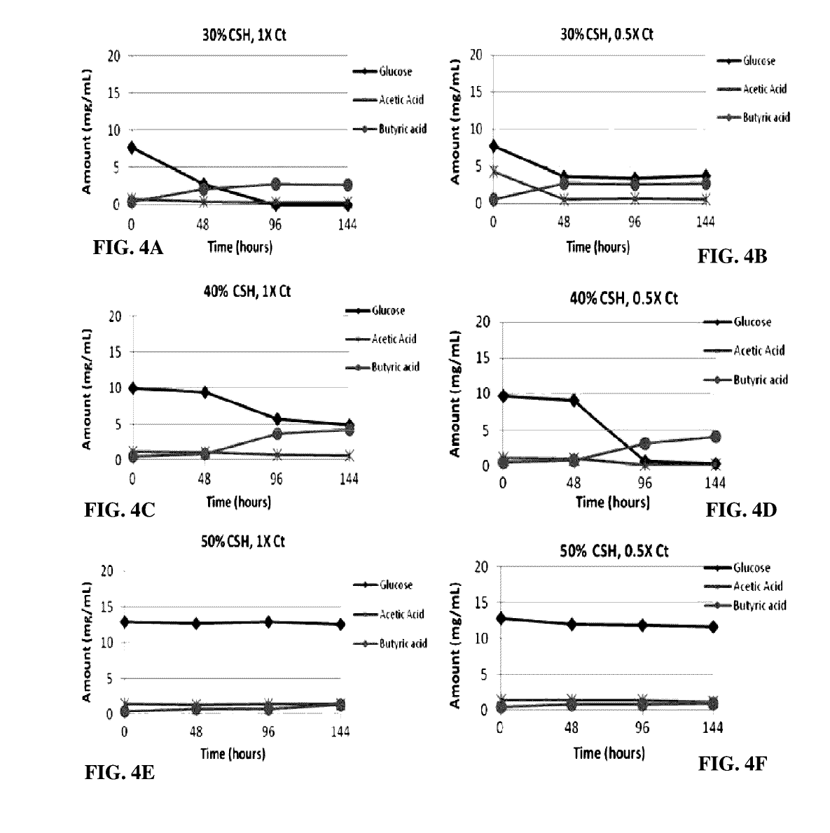

C. tyrobutyricum RPT-4213 was assessed in approximately 5 mL cultures for its capacity to grow and convert each of the five biomass hydrolysates into butyric acid. Biomass hydrolysates were tested at various concentrations of approximately 30%, 40%, or 50% (v/v) in Ct basal medium and half strength (approximately 0.5×) Ct. Wheat straw hydrolysate (WSH) appeared to be the best among those hydrolysates tested in supporting RPT-4213 growth and fermentation. As shown in FIG. 2, the fermentation broth containing approximately 50% WSH with 1× Ct produced the most butyric acid, approximately 8.06 g/L, with a yield of approximately 0.46 g of butyric acid per g of glucose which is approximately 95.8% of the theoretical yield (FIG. 2, bottom left panel). The anaerobic fermentation with approximately 50% SGH, 1× Ct produced approximately 6.01 g/L butyric acid, 0.44 g/g glucose (FIG. 3, bottom left panel) which is ranked next to WSH. Fermentation of approximately 50% CSH, 1× Ct (FIG. 4 bottom panels) resulted in a much slower growth and inefficient fermentation. At this concentration of CSH, the fermentation inhibitor HMF was present at approximately 4.38 mM (Table 2), which is approximately 133 μM per gram of glucose could be enough to inhibit growth and slow butyric acid fermentation. However, with approximately 40% CSH, 1× Ct, the butyric acid yield reached approximately 0.43 g/gram glucose (FIG. 4, middle left panel), suggesting that C. tyrobutyricum RPT-4213 may be able to ferment CSH at lower levels of inhibitors.

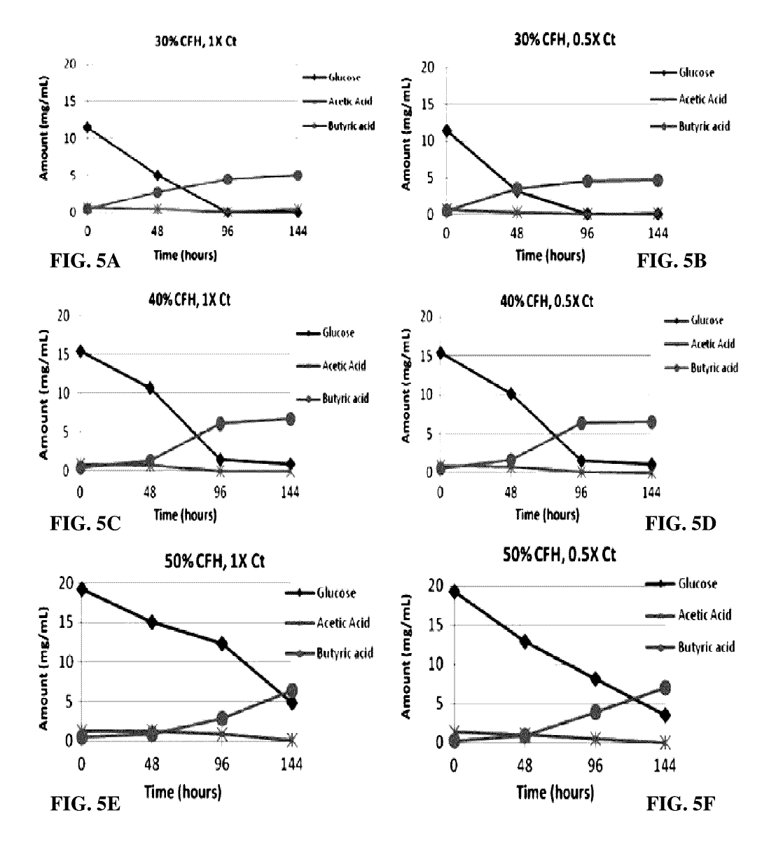

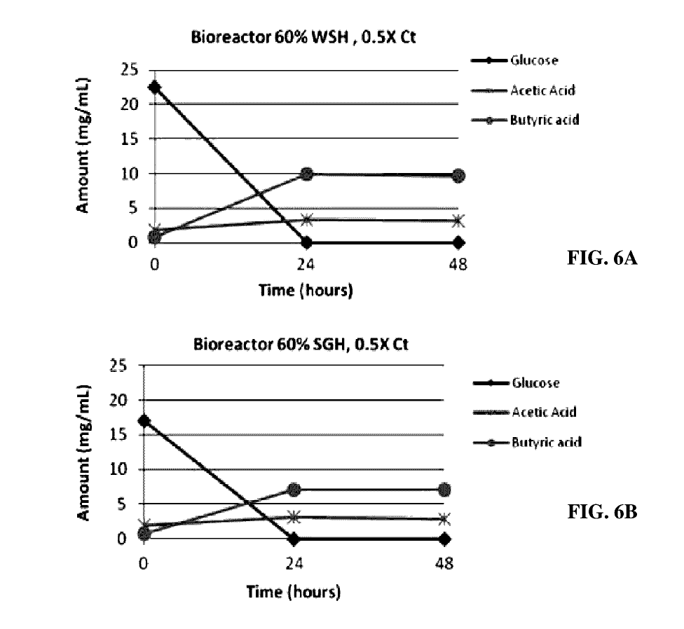

Results of approximately 5 mL culture fermentations of biomass substrates are summarized in Table 3. Overall, wheat straw hydrolysate appears to be the most suitable substrate for RPT-4213 growth and butyric acid production. Switchgrass hydrolysate (SGH) was a better substrate than CFH, CSH, and RHH (FIG. 5, Table 3). As shown in Table 2 CSH contained the highest level of HMF among substrates tested, and it appeared to be toxic for RPT-4213. High concentrations of a single inhibitor, such as HMF in CSH and furfural in CFH or a synergistic effect of a mixture of HMF and furfural could slow the growth and inhibit butyric acid production. Based on the results of the approximately 5 mL culture fermentations, wheat straw and switchgrass hydolysates were chosen for scale-up in pH-controlled mini-bioreactors. For these experiments, substrate concentrations of approximately 60% (v/v) were used in dilute (0.5×) Ct basal medium. As shown in FIG. 6, both WSH and SGH produced the highest levels of butyric acid within approximately 24 hours, much more quickly than in the 5 mL cultures without pH control. Maximal butyric acid concentrations were approximately 9.87 g/L from approximately 22.45 g/L glucose in WSH with a yield of approximately 0.44 g/g glucose. With SGH, approximately 7.05 g/L butyric acid was produced from approximately 16.98 g/L glucose with a yield of approximately 0.42 g/g glucose. The fermentation efficiencies were slightly lower than those observed in 5 mL cultures.

In the pH-controlled mini-bioreactor, the butyric acid fermentation appeared to be complete after about 24 hours. This suggests that pH control allows the fermentation to proceed much more quickly, although final conversion rates are similar. The bioreactor fermentation data further confirmed that WSH appears to be a superior substrate compared with SGH. Based on the amount of inhibitors released from approximately 100% hydrolysate (Table 2), the approximately 60% SGH broth contains approximately 147.0 μM HMF/g glucose and approximately 107.8 μM furfural/g glucose, a concentration that may have reduced the conversion efficiency without slowing the fermentation.

Under conditions tested, the titer of butyric acid produced by C. tyrobutyricum strain RPT-4213 from biomass is lower than in some previous reports (He et al., 2005; Jiang et al, 2011, 2009), although the yields of butyric acid per gram glucose are remarkably higher in the present invention. One reason is that relatively low concentrations of glucose, equal or less than approximately 20 g/L were used. Another reason is that batch fermentations were tested here, instead of using fed-batch or continuous fermentation systems or employing simultaneous product removal techniques, which can reduce inhibition of butyric acid fermentation by high concentrations of both substrate and product.

TABLE 3
Production, yield, and efficiency of butyric acid fermentation
from biomass hydolysates in 5 mL cultures of
C. tyrobutyricum strain RPT-4213.

| | Butyric | Butyric | Percentage of |
|---|---|---|---|
| Fermentation Broth | Acid g/L | Acid/glucose g/g | theoretical |
| 50% WSH, 1 X Ct | 8.06 | 0.46 | 95.8% |
| 50% SGH, 1 X Ct | 6.01 | 0.44 | 89.8% |
| 40% CFH, 1 X Ct | 4.34 | 0.44 | 89.8% |
| 40% CSH, 1 X Ct | 4.24 | 0.43 | 87.8% |
| 50% CFH, 1 X Ct | 6.45 | 0.34 | 69.4% |
| 50% RHH, 1 X Ct | 1.70 | 0.32 | 65.3% |

The foregoing detailed description and certain representative embodiments and details of the invention have been presented for purposes of illustration and description of the inventions. It is not intended to be exhaustive or to limit the invention to precise forms disclosed. It will be apparent to practitioners skilled in the art that modifications and variations may be made therein without departing from the scope of the invention.

What is claimed is:

1. A method for producing butyric acid comprising fermenting a lignocellulosic biomass hydrolysate with a C. tyrobutyricum strain.

2. The method of claim 1 wherein the C. tyrobutyricum strain is RPT-4213.

3. The method of claim 1 wherein the lignocellulosic biomass hydrolysate is made from agricultural biomass residue selected from the group consisting of wheat straw, corn fiber, corn stover, rice hull, and switchgrass.

4. The method of claim 3 wherein the lignocellulosic biomass hydrolysate is wheat straw hydrolysate.

5. A composition for use in a method for producing butyric acid comprising a seed culture of C. tyrobutyricum RPT-4213 and a fermentation medium.

6. A system for producing butyric acid comprising a bioreactor, a seed culture composition comprising C. tyrobutyricum RPT-4213, a lignocellulosic hydrolysate.

7. The system of claim 6 wherein said lignocellulosic hydrolysate is selected from the group consisting of wheat straw, corn fiber, corn stover, rice hull, and switchgrass.
